(19) United States
(12) Patent Application Publication
(10) Pub. No.: US 2021/0401309 A1
(43) Pub. Date: Dec. 30, 2021

(54) SYSTEM FOR DETERMINING AN ARTERIAL PULSE WAVE VELOCITY

(71) Applicants: HOSPICES CIVILS DE LYON, Lyon (FR); UNIVERSITÉ CLAUDE BERNARD LYON 1, Villeurbanne (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR); UNIVERSITÉ JEAN MONNET SAINT ETIENNE, Saint Etienne (FR)

(72) Inventors: Pierre Lantelme, Fontaines Saint Martin (FR); Andrei Cividjian, Meyzieu (FR); Brahim Harbaoui, Bron (FR)

(21) Appl. No.: 17/292,081

(22) PCT Filed: Oct. 31, 2019
(86) PCT No.: PCT/EP2019/079914

§ 371 Date: May 7, 2021

(30) Foreign Application Priority Data
Nov. 9, 2018 (EP) 18205481.7

(51) Int. Cl. A61B 5/021; A61B 5/318; A61B 5/339; A61B 5/00; A61B 5/0215

(52) U.S. Cl. CPC A61B 5/02108; A61B 5/318; A61B 5/339; A61B 2562/0247; A61B 5/02158; A61B 5/6851; A61B 5/7405

(57) ABSTRACT

A system for determining a pulse velocity wave comprises an interface for receiving a signal indicating the proximal blood pressure in an artery and for receiving a signal indicating distal blood pressure in the artery. A processing device is configured to determine a proximal rising edge between a diastolic pressure and the systolic pressure of the proximal signal; determine a proximal pressure peak prior to the proximal rising edge; determine a distal rising edge between a diastolic pressure and a systolic pressure of the distal signal; determine a distal pressure peak prior to the distal rising edge and to determine whether the distal pressure peak is in phase advance with respect to the proximal pressure peak; and determine a propagation velocity of a regressive pulse wave depending on the phase advance of the distal pressure peak.

### CROSS-REFERENCE TO RELATED APPLICATIONS

This application is a national phase entry under 35 U.S.C. § 371 of International Patent Application PCT/EP2019/079914, filed Oct. 31, 2019, designating the United States of America and published as International Patent Publication WO 2020/094509 A1 on May 14, 2020, which claims the benefit under Article 8 of the Patent Cooperation Treaty to European Union Patent Application Serial No. 18205481.7, filed Nov. 9, 2018.

### TECHNICAL FIELD

The disclosure relates to systems for assisting with the study of arterial pathologies, and, for example, to systems for anticipating a risk of rupture of an atheromatous plaque inside a coronary artery, with a view to refining the strategy with which a patient is managed during an angiocardiography examination, or to systems for studying pathologies in aortic arteries, renal arteries, or hepatic arteries, and more generally any artery in which there is a risk of rupture of an atheromatous plaque and/or thrombosis.

### BACKGROUND

It is known that calcification of an artery causes it to harden. Various techniques for estimating arterial stiffness are known: measurement of pulse pressure, estimation of arterial calcification, and pulse wave velocity (the latter technique being the most used). Studies have confirmed, for example, that the stiffness of the aortic artery, measured by various techniques, is an indicator that improves the prediction of cardiovascular pathologies. A medical study has shown that pulse wave velocity inside a coronary artery is lower in patients presenting acute coronary syndrome, possibly due to plaque rupture, than in patients without this pathology.

Although systems allowing aortic pulse-wave-velocity measurements to be taken, and therefore corresponding studies to be carried out, already exist, it is still tricky to accurately determine a coronary pulse wave velocity. Practitioners therefore find it difficult to measure coronary pulse wave velocity and thus to determine the impact of coronary stiffness on the progression of a coronary lesion, such as the risk of acute thrombosis, for example. Furthermore, determining aortic pulse wave velocity has proven to be insufficient to accurately determine the pathologies present in coronary arteries. In particular, measuring aortic pulse wave velocity does not allow the risk of rupture of an intracoronary plaque to be predicted.

The document ‘A Coronary Pulse Wave Velocity Measurement System’, published by Taewoo Nam et al., pages 975 to 977 in Proceedings of the 29th Annual International Conference of the IEEE EMBS, in the framework of a conference at the Cité Internationale de Lyon in France from 23 to 26 Aug. 2007, describes an example of a method for calculating, based solely on manual calculations, coronary pulse wave velocity on an experimental basis.

The document ‘Development of Coronary Pulse Wave Velocity: New Pathophysiological Insight Into Coronary Artery Disease’, published by Brahim HARBAOUI et al. in the Journal of the American Heart Association, volume 6, No. 2, 2 Feb. 2017, on pages 1 to 11, describes a method for determining a coronary pulse wave velocity, based on the time separating respective rising edges, between the diastolic and systolic pressures, of a signal of proximal blood pressure in a coronary artery and of a signal of distal blood pressure in the same coronary artery. This publication proposes a method that improves the precision with which the rising edges are identified. A distal rising edge is notably identified by an offset with respect to a distal falling edge.

The publication patent application EP3251591 describes a method for determining a coronary pulse wave velocity, based on the time separating the respective rising edges, between the diastolic and systolic pressures, of a signal of proximal blood pressure in a coronary artery and of a signal of distal blood pressure in the same coronary artery. This publication proposes a method that improves the precision with which the rising edges are identified. A distal rising edge is notably identified by an offset with respect to a distal falling edge.

In practice, the rising edges of blood-pressure signals may be difficult to identify. Specifically, peaks in arterial pressure may appear before rising pressure edges. When such pressure peaks appear, they interfere with the identification of the rising edges and the computation of arterial pulse wave velocity. Furthermore, arterial stiffness may vary between a compression phase and a decompression phase.

### BRIEF SUMMARY

The disclosure aims to overcome one or more of the aforementioned drawbacks. The disclosure thus relates to a system for determining a pulse wave velocity according to claim 1.

The disclosure also relates to the variants of the dependent claims. Those skilled in the art will understand that each of the features of the variants of the dependent claims may be independently combined with the features of the independent claim, without, however, constituting an intermediate generalization.

### BRIEF DESCRIPTION OF THE DRAWINGS

Other features and advantages of the disclosure will become clearly apparent from the completely non-limiting description thereof that is given below, by way of indication, with reference to the accompanying drawings, in which:

FIG. 1 is a schematic representation of a heart and its coronary arteries;

FIG. 2 is a cross-sectional view of a guidewire according to one aspect of the disclosure, which guidewire is inserted into a coronary artery comprising a stenosis;

FIG. 3 is a schematic cross-sectional view of an FFR guidewire device according to one aspect of the disclosure (FFR being the acronym of fractional flow reserve);

FIG. 4 is a schematic representation of a system for processing signals with a view to determining pulse wave velocity and the ischemic character of a coronary stenosis according to one aspect of the disclosure;

FIG. 5 is a graph illustrating an example of a proximal-coronary-arterial-pressure cycle;

FIG. 6 is a graph illustrating an example of a distal-coronary-arterial-pressure cycle;

FIG. 7 illustrates temporal parameters in the vicinity of the rising edge of a proximal-coronary-arterial pressure and of a distal-coronary-arterial pressure; and

FIG. 8 illustrates an example of determination of temporal parameters in the vicinity of the rising edge of a proximal-coronary-arterial pressure and of a distal-coronary-arterial pressure.

### DETAILED DESCRIPTION

The inventors have observed that pressure peaks may appear in the intra-coronary pressure signals measured both in the proximal and in the distal position, prior to the rising edges between the diastolic pressure and the systolic pressure. The inventors' interpretation is that such early pressure peaks are due to a backward wave, i.e., one travelling in the direction opposite to the direction of blood flow (i.e., from the distal coronary end to the proximal coronary end). Such peaks in arterial pressure are due to a pressure exerted from outside the artery, for example by other parts of the body or by an external object. The backward wave may, for example, be caused by a compression of the distal end of the coronary artery by the myocardium during cardiac contraction. Surprisingly, the inventors have identified that analysis of such early pressure peaks may be exploited to determine the velocity of the pulse wave in the coronary artery,

The disclosure provides a system for digitally computing a pulse wave velocity, based on analysis of the identified backward wave. The disclosure is applicable, in particular, to the computation of an arterial pulse wave velocity when an external pressure may prevent the rising pressure edge from being detected accurately, and, in particular, to the computation of a coronary pulse wave velocity.

The disclosure allows the pulse wave velocity to be accurately and reproducibly determined, thereby facilitating decision-making by the practitioner, with a view to determining how the patient will be managed, in cases where a backward pulse wave decreases the ability to analyze rising edges of blood-pressure signals. In addition, in the case of a coronary artery, the disclosure may be implemented at the same time as the already clinically validated procedure for introducing a guidewire with a view to measuring FFR index.

FIG. 1 is a schematic representation of a human heart 1. The aortic artery 11, which is connected to the heart, and coronary arteries 12 to 15 may be seen. The coronary arteries are intended to supply oxygenated blood to the heart muscles. FIG. 1 notably illustrates the right coronary artery 12, the posterior descending coronary artery 13, the left circumflex coronary artery 14 and the left anterior descending coronary artery 15. The disclosure will be described here in the context of a particular application to a coronary artery, but it will possibly be implemented with other types of arteries.

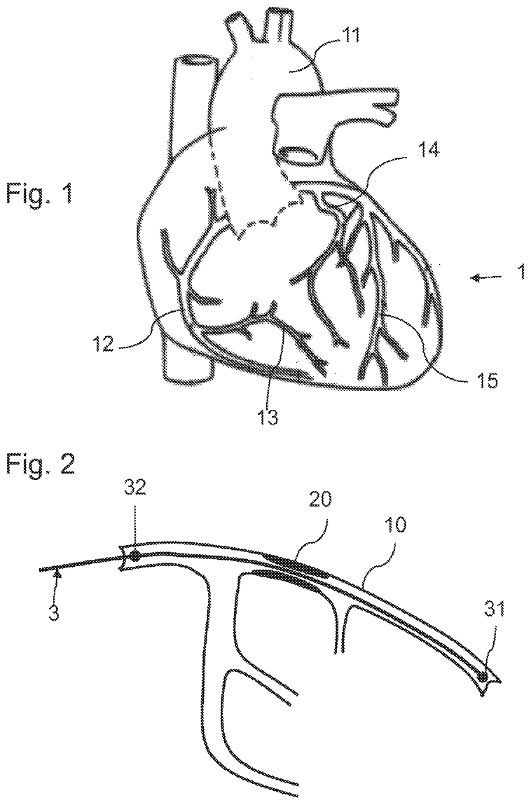

FIG. 2 illustrates an example of a method for retrieving signals with a view to computing the coronary pulse wave velocity of a patient. An FFR guidewire 3 is inserted so as to position its free end inside a coronary artery 10. The guidewire 3 here comprises two pressure sensors 31 and 32 at its free end. The terms distal and proximal will refer to the relative proximity of a point in question, with respect to the blood flow coming from the heart. The pressure sensor 31 is in a distal position, in order to measure the blood pressure in proximity to the junction of the coronary artery 10 with the tissue of to capillaries. The pressure sensor 32 is in a proximal position, in order to measure the blood pressure in proximity to the junction of the coronary artery 10 with the aortic artery. The pressure sensor 32 is a predefined distance Dmd from the pressure sensor 31 along the length of the guidewire 3. The coronary artery 10 illustrated here comprises a stenosis 20, and the pressure sensors 31 and 32 are positioned on either side of this stenosis 20.

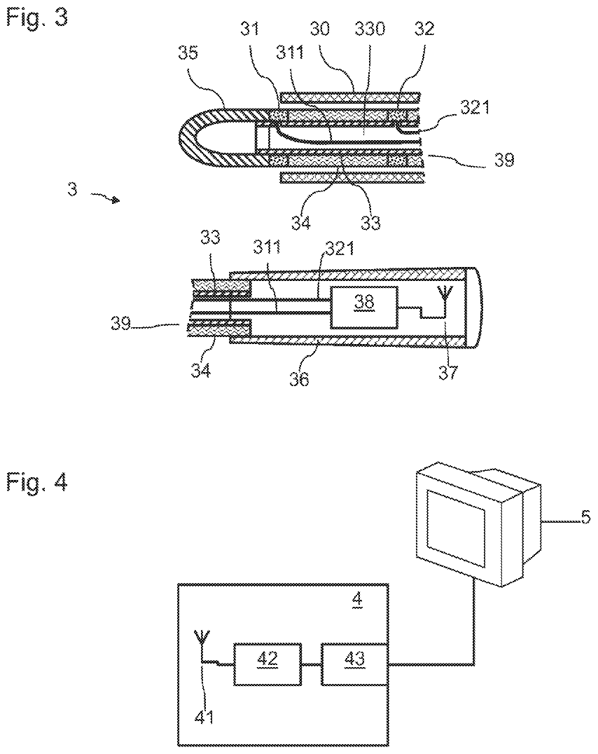

FIG. 3 is a schematic cross-sectional view of two ends of a guidewire 3 that may be used to implement the disclosure. The guidewire 3 comprises a wire 39 that slides in a way known per se through an outer storage sheath 30. The wire 39 is only schematically illustrated, in order to show its structure; the wire 39 has not been drawn to scale. The wire 39 is flexible in order to adapt to the morphology of the coronary artery into which it is inserted. The wire 39 comprises a hollow metal sleeve 33. The metal sleeve 33 is covered with a sheath 34 made of synthetic material. The wire 39 advantageously comprises an end fitting 35 at its free end. The end fitting 35 may advantageously be flexible and radiopaque. The end fitting 35 is here attached to the metal sleeve 33.

The pressure sensor 31 is here attached to the periphery of the sleeve 33, and positioned between the end fitting 35 and the sheath 34. The pressure sensor 31 is intended to measure the distal blood pressure. The pressure sensor 31 (of a structure known per se) is connected to a cable or to an optical fiber 311 for transmitting the pressure signal. The cable or optical fiber 311 passes through an aperture in the sleeve 33 with a view to connection thereof to the pressure sensor 31. The cable or optical fiber 311 extends into an internal bore 330 of the sleeve 33.

The pressure sensor 32 is here attached to the periphery of the sleeve 33, and positioned between two segments of the sheath 34. The pressure sensor 32 is intended to measure the proximal blood pressure. The pressure sensor 32 is connected to a cable or to an optical fiber 321 for transmitting the pressure signal. The cable or optical fiber 321 passes through an aperture in the sleeve 33 with a view to connection thereof to the pressure sensor 32. The cable or optical fiber 321 extends into the internal bore 330 of the sleeve 33.

The wire 39 is here flexible but substantially non-compressible or inextensible. Thus, the wire 39 here maintains a constant distance Dmd between the pressure sensors 31 and 32. The distance between the pressure sensors 31 and 32 corresponds in practice to the curvilinear distance between these sensors along the wire 39. The distance between the pressure sensors 31 and 32 is advantageously at least equal to 50 mm, so as to guarantee that the distance between these pressure sensors 31 and 32 is large enough to provide a high level of accuracy for the pulse-wave-velocity computation. Moreover, the distance between the pressure sensors 31 and 32 is advantageously at most equal to 200 mm, so that the guidewire 3 remains usable in most coronary arteries of standard length, Moreover, using a guidewire 3 comprising pressure sensors 31 and 32 that are held at a predefined distance allows inaccuracies related to the distance between two pressure measurements inside a coronary artery to be removed.

Opposite its free end, the wire 39 is attached to a handle 36. The sleeve 33 and the sheath 34 are here embedded in the handle 36. The handle 36 thus allows the wire 39 to be moved. in this example, the guidewire 3 is configured to deliver the measured pressure signals to a processing system via a wireless interface. However, it is also possible to envision the guidewire 3 communicating with a processing system via a wired interface. A digitization and driving circuit 38 is here housed inside the handle 36. The cables or optical fibers 311 and 321 of the wire 39 are connected to the circuit 38. The circuit 38 is connected to a transmitting antenna 37. The circuit 38 is configured to digitize the signals measured by pressure sensors 31 and 32 and delivered by the cables or optical fibers 311 and 321. The circuit 38 is also configured to transmit, via the transmitting antenna 37, using a suitable communication protocol, the digitized signals to a remote location. The circuit 38 is supplied with electrical power in a way known per se and that will not be described here.

The sheath 34 may be made of a hydrophobic material at the free end of the wire 39, and may be made of another material such as PTFE (polytetrafluoroethylene) between the free end and the handle 36.

Using an FFR guidewire 3, use of which has been approved by health authorities and forms part of routine clinical practice, allows a system 4 according to the disclosure to be used with a substantially streamlined clinical validation process.

The guidewire 3 communicates with a signal-processing system 4. The system 4 here comprises a wireless communication or receiving interface 41 with the guidewire 3. However, it is also conceivable for the guidewire 3 to communicate with a processing system 42 (also referred to herein as a “processing device 42” and/or a “processing circuit 42”) via a wired interface. The system 4 thus comprises a receiving antenna forming a receiving interface 41 (also referred to herein as a “receiving antenna 41”) that is configured to receive the information communicated by the transmitting antenna 37. The receiving antenna 41 is connected to the processing circuit 42, a computer for example. The system 4 comprises a wired communication interface 43, The interface 43, for example, allows the results computed by the processing circuit 42 to be displayed on a display screen 5. An anti-aliasing filter and an analog/digital converter may, for example, be integrated into the processing circuit 42, or into the guidewire 3, in order to allow the processing circuit 42 to process the digital proximal- and distal-coronary-blood-pressure signals.

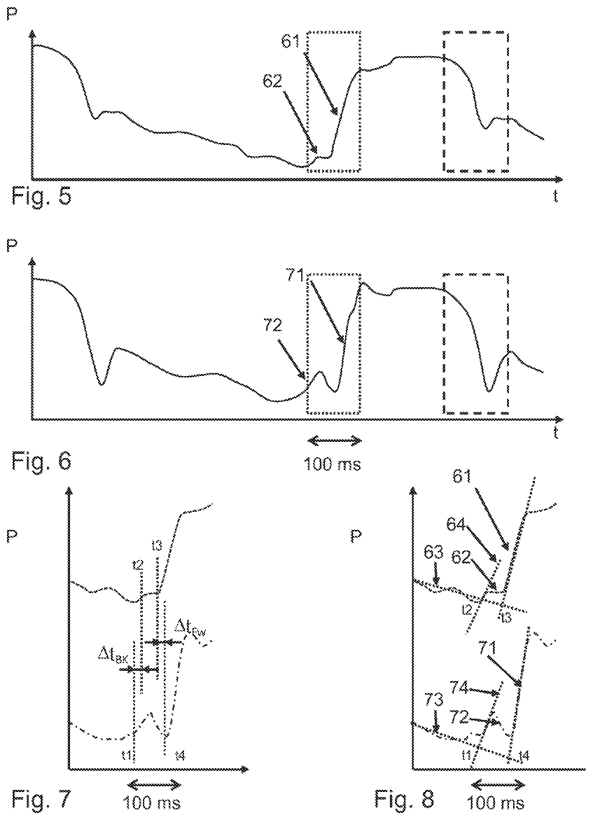

FIG. 5 is a graph illustrating an example of a proximal-coronary-arterial-pressure cycle, and FIG. 6 is a graph illustrating an example of a distal-coronary-arterial-pressure cycle. In a compression phase, illustrated in the dotted window, the arterial pressures change from a diastolic pressure value to a systolic pressure value. In the compression phase, the proximal pressure comprises a rising edge 61, which is preceded by a pressure peak 62. The pressure peak 62 has an amplitude lower than the amplitude of the rising edge 61 (the latter amplitude being equal to the proximal systolic pressure minus the proximal diastolic pressure). In a decompression phase, illustrated in the dashed window, the proximal arterial pressures change from a systolic pressure value to a lower pressure value, with a nadir when the aortic valve closes (moment of the appearance of the dicrotic notch). On the distal side of the coronary artery, during the compression phase, the distal pressure comprises a rising edge 71, which is preceded by a pressure peak 72. The pressure peak 72 has an amplitude lower than the amplitude of the rising edge 71 (the latter amplitude being equal to the distal systolic pressure minus the distal diastolic pressure). In a decompression phase, illustrated in the dashed window, the distal arterial pressures change from a systolic pressure value to a lower pressure value, with a nadir when the aortic valve closes (moment of the appearance of the dicrotic notch).

FIG. 7 illustrates temporal parameters in the vicinity of the rising edge of a proximal-coronary-arterial pressure and of a distal-coronary-arterial pressure. From the arterial-pressure signals measured in the proximal position (top curve) and in the distal position (bottom curve), temporal parameters may be determined. It may be seen that the pressure peak 72 begins at the time t1, that the pressure peak 62 begins at the time t2, that the rising edge 61 begins at the time t3 and that the rising edge 71 begins at the time t4. It may be seen that the time t1 precedes the time t2 by a value Δt_{BK}. It may be seen that the time t3 precedes the time t4 by a value Δt_{FW}.

FIG. 8 illustrates an example of the extrapolation of the pressure curves at the times t1 to t4 that may be carried out by the processing device 42, on the basis of the arterial-pressure signals. The time t2 is, for example, defined to be the time corresponding to the intersection between a straight line (or alternatively an exponential curve, or a curve according to another law) representative of the decrease in diastolic pressure (straight line 63) and a straight line 64 corresponding to the pressure rise of the peak 62. The time t3 is, for example, defined to be the time corresponding to the intersection between the straight line 63 and the straight line corresponding to the rising edge 61. The time t1 is, for example, defined to be the time corresponding to the intersection between a straight line (or alternatively an exponential curve, or a curve according to another law) representative of the decrease in diastolic pressure (straight line 73) and a straight line 74 corresponding to the pressure rise of the peak 72. The time t4 is, for example, defined to be the time corresponding to the intersection between the straight line 73 and the straight line corresponding to the rising edge 71. As the distal peak 72 is in phase advance with respect to the proximal peak 62, a backward coronary pulse wave the velocity of which is equal to Dmd/Δt_{BK} is indeed present. The velocity of the forward pulse wave, which is determined via the separation between the proximal rising edge 61 and the distal rising edge 71, is equal to Dmd/Δt_{FW}. According to the disclosure, the pulse wave velocity is based on the backward pulse wave.

In a study carried out on healthy animal test subjects (anesthetized pigs) it was observed that forward pulse wave velocity and backward pulse wave velocity are strongly correlated (r²=0.83, n=10) under baseline conditions (spontaneous arterial pressure and heart rate). In the presence of a coronary stenosis (inflation of an angioplasty balloon between the proximal and distal positions with a cross-sectional area approximately equal to half the cross-sectional area of the artery as measured using the IVUS technique (IVUS being the acronym of intravascular ultrasound)) computation of pulse wave velocity based on the backward pulse wave proves to be more reliable than computation based on the forward pulse wave. The ratio between the amplitude of the backward pulse wave and the forward pulse wave was also found to increase with the severity of the stenosis. The more severe and substantial this stenosis, the greater the inaccuracy of the computation of pulse rate based on the forward wave, and the greater the accuracy of the computation of pulse rate based on the backward wave. Thus, the accuracy level of a system for computing pulse wave velocity according to the disclosure increases with the severity of the pathology.

The operation of the system 4 for computing pulse wave velocity will now be detailed. The receiving interface 41 is configured to receive the proximal-blood-pressure signal and the distal-blood-pressure signal for an artery, either in a post-processing mode or directly from the pressure sensors 31 and 32.

The processing device 42 is configured, in a way known per se, to determine a proximal rising edge between a diastolic pressure and a systolic pressure of the proximal-blood-pressure signal. The proximal rising edge corresponds to an increase in proximal pressure between the proximal diastolic pressure and the proximal systolic pressure. The processing device 42 is thus configured to determine the time t3 detailed above. The processing device 42 is also configured, in a way known per se, to determine a distal rising edge between a diastolic pressure and a systolic pressure of the distal-blood-pressure signal. The distal rising edge corresponds to an increase in distal pressure between the distal diastolic pressure and the distal systolic pressure. The processing device 42 is thus configured to determine the time t4 detailed above.

It is possible, for example, to envision sampling a distal pressure and/or a proximal pressure at a frequency comprised between 500 Hz and 5 kHz. For a sampling frequency that is deemed insufficient, it is possible to interpolate the sampling values (for example, using cubic splines), then to sample the interpolated signal anew at a frequency higher than the initial sampling frequency (oversampling). For example, for a sampling frequency of 500 Hz, it is possible to envision oversampling the interpolated signal at a frequency of 2 kHz or more.

The processing device 42 is also configured to determine the proximal pressure peak 62 prior to the proximal rising edge 61, during a phase of decrease in proximal diastolic pressure. The processing device 42 is thus configured to determine the time t2 detailed above. The processing device 42 is furthermore configured to determine the distal pressure peak 72 prior to the distal rising edge 71, during a phase of decrease in distal diastolic pressure. The processing device 42 is thus configured to determine the time t1 detailed above. The processing device 42 will possibly be configured to search for a pressure peak in a time window of a duration between 50 and 100 ms before the corresponding rising edge.

The processing device 42 is also configured to determine the amplitude of the pressure peaks. If a plurality of pressure peaks is identified in this time window, the processing device 42 selects the pressure peak having the highest amplitude. The identification of a pressure peak may be dependent on a peak having an amplitude higher than a set threshold or higher than a predefined proportion of the pulsed pressure (difference between the systolic pressure and the diastolic pressure).

The processing device 42 then determines the propagation velocity of the backward pulse wave depending on a phase advance of the distal pressure peak with respect to the determined proximal pressure peak. In particular, the propagation velocity VOPr of the backward pulse wave may be found using the following relationship: VOPr=(t2−t1)/Dmd. This relationship is based on the exploitation of a time reference received via the receiving interface 41 for the proximal-blood-pressure signal and for the distal-blood-pressure signal, respectively.

The distance Dmd may be either a set value corresponding to a predetermined distance between the pressure sensors 31 and 32 (value, for example, stored in the guidewire 3 or in the system 4), or a value of a movement of a single sensor, with which pressure measurements are carried out sequentially, separated by the distance Dmd. It is also possible to make provision to use an FFR guidewire equipped with a single pressure sensor, which is moved by the practitioner a predefined distance between the distal position and the proximal position in the studied artery. During the analysis of the respective pressure signals in the proximal position and in the distal position, this distance Dmd is taken into account to compute the pulse wave velocity.

The receiving interface 41 may also be configured to receive a time indicator of a synchronization event chosen from an isovolumic cardiac contraction and an opening of the aortic valve of the heart connected to the artery to be analyzed. The receiving interface 41 may also be configured to receive an electrocardiogram signal, an audio signal or an imaging signal relating to the heart connected to the artery to be analyzed. Thus, in the case where the proxi pressure and distal-pressure signals are not simultaneous, they may be synchronized with a common reference signal or a common synchronization event relating to the patient's heart.

When the processing device 42 is unable to identify a pressure peak prior to its respective rising edge, it implements a pulse-wave-velocity computation based on the forward pulse wave, for example as detailed in the document EP3251591.

Advantageously, the processing device 42 may be configured to receive information on the position of the site of measurement of pressure in the artery. The processing device 42 may then be configured to determine a reference pressure-sensor position, from which the backward waves appear or disappear. The processing device 42 may be configured to compute the backward wave velocity for a plurality of positions on the basis of the reference position. The processing device 42 will be able to select or retain the backward-wave-velocity value computed for the position furthest away from the reference position.

The processing device 42 may determine the times t3 and t4 using methods other than those described above. In particular, the processing device 42 may compute the first or second derivative of a proximal and/or distal pressure, then determine the times at which this first or second derivative crosses a positive threshold and a negative threshold, respectively, in order to identify the corresponding edge. The processing device 42 may determine the times t1 and t2 using methods other than those described above. In particular, the processing device 42 may compute the first or second derivative of a proximal and/or distal pressure, then determine the times at which this first or second derivative crosses a positive threshold and a negative threshold, respectively, in order to identify the corresponding pressure peak.

Advantageously, the processing circuit 42 may implement low-pass filtering (for example, with a cutoff frequency between 10 and 20 Hz), to remove the rapid pressure fluctuations between heart beats, before determining the presence of the pressure peaks and the times of their appearance.

The computed backward pulse wave velocity may be compared to a reference threshold for a similar artery and patient. When the computed backward pulse wave velocity crosses such a reference threshold (a low threshold or a high threshold, as appropriate), the processing circuit 42 will possibly generate a suitable warning signal in order to draw the attention of a practitioner. Various thresholds will possibly be used, notably depending on various risk factors such as hypertension, diabetes, dyslipidemia, smoking habits, family history of coronary cardiovascular problems, a prior coronary cardiovascular episode, or the composition of the atheromatous plaque as estimated using medical-imaging methods.

1. A system for determining a pulse wave velocity, comprising:
an interface for receiving a signal of proximal blood pressure in an artery and for receiving a signal of distal blood pressure in the artery; and
a processing device configured to:
determine a proximal rising edge between a diastolic pressure and a systolic pressure of the signal of the proximal blood pressure;
determine a proximal pressure peak prior to the proximal rising edge;
determine a distal rising edge between a diastolic pressure and a systolic pressure of the signal of the distal blood pressure;
determine a distal pressure peak prior to the distal rising edge and to determine whether the distal pressure peak is in phase advance with respect to the proximal pressure peak; and
determine a propagation velocity of a backward pulse wave depending on the phase advance of the distal pressure peak with respect to the proximal pressure peak.

2. The system of claim 1, wherein the interface is configured to receive a time reference for the signal of the proximal blood pressure and for the signal of the distal blood pressure, the processing device being configured to determine the propagation velocity of the backward pulse wave depending further on a temporal offset between the distal pressure peak and the proximal pressure peak.

3. The system of claim 1, wherein the interface is configured to receive a time indicator of a synchronization event selected from an isovolumic cardiac contraction and opening of an aortic valve of a heart connected to the artery.

4. The system of claim 1, wherein the interface is configured to receive an electrocardiogram signal, an audio signal, or an imaging signal from a heart connected to the artery.

5. The system of claim 1, wherein the interface is configured to receive a position of a pressure sensor, the processing device being further configured to determine a reference pressure-sensor position in which the backward pulse wave disappears.

6. The system of claim 1, wherein the processing device is further configured to determine the amplitude of the distal pressure peak.

7. The system of claim 6, wherein the processing device is further configured to determine the presence of the distal pressure peak when the amplitude of the distal pressure peak exceeds a predefined threshold.

8. The system of claim 6, wherein the processing device is further configured to compute a ratio between the amplitude of the distal pressure peak and the distal rising edge.

9. The system of claim 1, wherein the processing device is further configured to:
identify respective phases of decrease in diastolic pressure in the signal of the proximal blood pressure and in the signal of the distal blood pressure; and
identify a beginning of the rising edges of the distal and proximal blood pressures by determining an intersection between the identified respective phases of decrease in diastolic pressure and respective tangents to the rising edges of the distal and proximal blood pressures.

10. The system of claim 1, wherein the processing device is further configured to:
identify respective phases of decrease in diastolic pressure in the signal of the proximal blood pressure and in the signal of the distal blood pressure; and
identify a beginning of the peaks of the distal and proximal blood pressures by determining an intersection between the identified respective phases of decrease in diastolic pressure and respective tangents to the peaks of the distal and proximal blood pressures.

11. The system of claim 1, wherein:
the interface is configured to retrieve a value of the distance between a site of measurement of the proximal blood pressure and a site of measurement of the distal blood pressure, and
the processing device is configured to determine the propagation velocity of the backward pulse wave depending further on the retrieved value of the distance.

12. The system of claim 1, further comprising an elongate FFR guidewire comprising two pressure sensors offset by a predefined distance along the length of the guidewire, the two pressure sensors being connected to the interface, the interface comprising a circuit for sampling respective signals of the two pressure sensors.

13. The system of claim 12, wherein:
the interface is configured to receive information on positions of the two pressure sensors in the artery; and
the processing device is further configured to:
determine a reference pressure-sensor position in which the backward pulse wave disappears;
store the propagation velocity of the backward pulse wave for a plurality of the positions of the two sensors away from the determined reference pressure-sensor position, and
select the propagation velocity of the backward pulse wave for the position, of the plurality of the positions of the two sensors, that is furthest away from the determined reference pressure-sensor position.
